(19) United States
(12) Patent Application Publication
(10) Pub. No.: US 2022/0142600 A1
(43) Pub. Date: May 12, 2022

(54) Systems and Methods for Sound Mapping of Anatomical and Physiological Acoustic Sources Using an Array of Acoustic Sensors

(71) Applicant: The Medical College of Wisconsin, Inc., Milwaukee, WI (US)

(72) Inventors: Brandon Tefft, Milwaukee, WI (US); Shayan Shafiee, Milwaukee, WI (US)

(21) Appl. No.: 17/427,005

(22) PCT Filed: Jan. 31, 2020
(86) PCT No.: PCT/US2020/016179

§ 371 Date: Jul. 29, 2021

Related U.S. Application Data
(60) Provisional application No. 62/799,364, filed on Jan. 31, 2019.

(51) Int. Cl. A61B 7/00; H04R 29/00; H04R 1/32; H04R 3/00; G10L 19/02; A61B 5/00; A61B 5/339; A61B 5/0205

(52) U.S. Cl. CPC A61B 7/00; H04R 29/008; H04R 1/326; H04R 3/005; G10L 19/0216; H04R 2201/401; A61B 5/339; A61B 5/0205; A61B 5/0022; H04R 2420/07; A61B 5/6831

(57) ABSTRACT

Described here are systems and methods for generating sound maps that depict the spatiotemporal distribution of sounds occurring within a subject. To this end, the sound maps may be four-dimensional (“4D”) maps that depict the three-dimensional spatial distribution of acoustic sources within a subject, and also the temporal evolution of sounds measured at those acoustic sources over a duration of time.

### CROSS-REFERENCE TO RELATED APPLICATIONS

This application claims the benefit of U.S. Provisional Patent Application Ser. No. 62/799,364, filed on Jan. 31, 2019, and entitled “SYSTEMS AND METHODS FOR SOUND MAPPING OF ANATOMICAL AND PHYSIOLOGICAL ACOUSTIC SOURCES USING AN ARRAY OF ACOUSTIC SENSORS,” which is herein incorporated by reference in its entirety.

### BACKGROUND

Early detection and diagnosis of disease is important for slowing or preventing disease progression, and offers the potential to save lives and reduce healthcare costs. Routine medical diagnostics can encourage patients to make healthy lifestyle choices and to address diseases at early stages when interventions are the most effective and least expensive. The stethoscope revolutionized medicine by allowing physicians to use sound to diagnose diseases of the heart, lungs, and intestines. Over 200 years later, the stethoscope remains a staple of medical practice, but more modern means of detecting sound are needed to unlock further diagnostic potential.

### SUMMARY OF THE DISCLOSURE

The present disclosure addresses the aforementioned drawbacks by providing a method for generating a sound map that depicts a spatial distribution of acoustic sources within a subject. The method includes acquiring acoustic signal data from a subject using an array of acoustic sensors coupled to a surface of the subject and arranged around an anatomical region-of-interest. Relative position data that indicate a relative position of acoustic sensors in the array of acoustic sensors are also provided. One or more sound maps are reconstructed from the acoustic signal data and using the relative position data. These sound maps depict a spatial distribution of acoustic sources in the subject at one or more time points.

It is another aspect of the present disclosure to provide a sound map generating system that includes a sensor array and a computing device in communication with the sensor array. The sensor array is configured to be worn around an anatomical region-of-interest of a subject, and includes a plurality of acoustic sensors and an elastic motion sensor coupling each of the acoustic sensors to form the sensor array. The computing device is configured to: receive acoustic signal data from the plurality of acoustic sensors; receive relative position data from the elastic motion sensor; and reconstruct from the acoustic signal data using the relative position data, a sound map that depicts a spatial distribution of acoustic sources in a subject wearing the sensor array.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for generating a sound map from acoustic signal data recorded from a subject.

FIG. 2 is an example of a system that can be used to record acoustic signal data and reconstruct sound maps from such data.

FIG. 3 is another example of a system that can be used to record acoustic signal data and reconstruct sound maps from such data.

FIG. 4 is a block diagram of an example system for generating one or more sound maps from acoustic signal data.

FIG. 5 is a block diagram illustrating example hardware components of the system of FIG. 4.

### DETAILED DESCRIPTION

Described here are systems and methods for generating sound maps that depict the spatiotemporal distribution of sounds occurring within a subject. To this end, the sound maps may be four-dimensional (“4D”) maps that depict the three-dimensional spatial distribution of acoustic sources within a subject, and also the temporal evolution of sounds measured at those acoustic sources over a duration of time. In other instances, the sound maps can be three-dimensional (“3D”) maps that depict the spatial distribution of acoustic sources at a single time point.

In this way, the systems and methods described in the present disclosure provide a modern, computerized stethoscope that can produce 3D and/or 4D mappings of sounds recorded from a subject over time and space. Such sound maps can be useful for diagnosing and/or monitoring diseases of the heart, lungs, respiratory tract, gastrointestinal tract, joints, and other organs, tissues, and anatomy. In some instances, the sound maps can also be monitored and/or analyzed to assess the efficacy of a particular treatment, such as a drug treatment.

As one non-limiting example, heart disease is a leading cause of morbidity and mortality in the developed world. Current diagnosis of heart disease typically requires an invasive catheterization procedure to visualize the narrowed arteries. Such invasive procedures are typically done once the disease is very advanced and requires aggressive treatment; thus, these procedures are not often used to aid early detection.

It is contemplated that other cardiac conditions and pathologies can also be detected and/or monitored by acquiring sound maps. As one non-limiting example, patients who have infectious endocarditis will have different cardiac sound signatures relative to healthy hearts and valves; thus, acquiring and monitoring sound maps can help detect this pathology. As another example, the sound maps can be used to detect and/or monitor anatomical cardiac abnormalities, including but not limited to “single ventricle anatomy,” valve abnormalities (e.g., bicuspid aortic valve defects), diastolic dysfunction (e.g., heart filling), heart failure with reduced ejection fraction or preserved ejection fraction, wall motion abnormalities, and so on. Advantageously, the 4D sound maps can be used to non-invasively detect such cardiac abnormalities in pediatric and other patients, such that early interventions can be provided.

Sound maps can also be acquired to measure or otherwise monitor cardiac conditions or function, such as monitoring atrial pressure (e.g., left atrial pressure), inflow (e.g., mitral valve inflow), atrial hypertension (e.g., left atrial hypertension), and so on. By monitoring pressure in a blood vessel, the systems and methods described in the present disclosure can provide a non-invasive alternative to blood pressure monitors that are implanted within a patient's blood vessels (e.g., arterial pressure monitors).

The systems and methods described in the present disclosure can be used to measure and monitor heart sounds. As one example, sound maps can be generated to catalog sound signatures of different cardiac sounds, including different heart murmur sounds. In this way, the sound maps can provide an alternative screening tool to identify patients, including pediatric patients, who have a murmur that should be further evaluated, such as with echocardiography or other diagnostic tools or procedures. In a similar way, sound maps can be recorded during exercise or activities of daily living. In addition to monitoring a patient's current condition (e.g., similar to stress echocardiography, monitoring for changes in an aortic aneurysm during activity) these data can be stored as training data for training machine learning algorithms, or to otherwise learn whether particular sound signatures can be attributable to specific conditions or problems.

As another non-limiting application, sound maps can be acquired in order to detect the point of maximal impulse (“PMI”). By tracking the PMI over time, it can be possible to detect whether the PMI is moving laterally, which may be indicative of a changing or otherwise undetected cardiac condition or pathology.

The sound maps generated using the systems and methods described in the present disclosure can be used to identify narrowing arteries at the onset of disease by virtue of the sound produced by resistance to blood flow. In some instances, the location of a stenosis within a blood vessel (e.g., renal artery stenosis, stenosis in other vessels) may be determined, or estimated, from a sound map of the region containing the stenosis. Routine screenings could be used to encourage at risk patients to adopt healthy lifestyle changes and mitigate the risk of the disease progressing to life-threatening stages. In a similar way the systems and methods described in the present disclosure can be used to monitor for thrombosis, such as shunt thrombosis. Blood flow in peripheral vasculature (e.g., in the legs) can also be monitored to detect ischemia, clotting, narrowing (e.g., intermittent claudication versus regular leg cramps), and so on. It is contemplated that the sound signatures measured from vasculature can be analyzed to detect and distinguish laminar blood flow from turbulent blood flow.

As noted above, sound maps can also be acquired from anatomical locations other than the heart and vasculature. For instance abdominal sounds can be mapped. As one example, abdominal sounds can be mapped in order to detect indistinct bowel sounds, lack of bowel sounds, and so on. The systems and methods described in the present disclosure can also be used to monitor swallowing in order to non-invasively detect dysfunction swallowing.

As another example, respiratory sounds can be mapped. For instance, sound maps can be used to separately map lung sounds. In this way, conditions and pathologies such as pneumonia, edema, chronic obstructive pulmonary disease (“COPD”), crackling, tumors, mucus plugs, and so on, can be detected and/or monitored. Similarly, pulmonary embolisms can be detected, including detecting and identifying which lung is affected.

As still another example, the systems and methods described in the present disclosure can have obstetric applications. For instance, sound maps can be acquired and used for monitoring signs of pre-eclampsia, fetal movement, fetal heart sounds, placental blood flow, and so on.

In general, an array of sensitive microphones or other acoustic measurement devices or sensors are placed around an anatomical region-of-interest (e.g., a subject's chest, abdomen, or both). Sound recordings are then captured for a period of time and a computer system is used to process the recordings into a 4D sound map. The sound map can be visualized on the computer system. As one example, the 4D sound map can depict the sound intensity in time and space being encoded by a spectrum of colors. Basic anatomy can be visible from any structures producing sound (e.g., heart chambers, heart valves, arteries, and veins). A user can visually inspect the sound maps for focal abnormalities in sound intensity, duration, location, or other indicators of disease. Additionally or alternatively, a computer system can analyze the sound maps to identify focal abnormalities in sound intensity, duration, location, or other indicators of disease. For instance, a machine learning algorithm trained on appropriate training data (e.g., sound maps obtained from a population and labeled by a user) can be used to automatically or semi-automatically analyze sound maps.

The systems and methods described in the preset disclosure fill a gap between the simple stethoscope and advanced diagnostics such as ultrasound, magnetic resonance imaging (“MRI”), computed tomography (“CT”), and angiography. These systems and methods can be used to supplement existing medical diagnostic technologies already in use by healthcare providers. Advantageously, the systems and methods are inexpensive and safe enough to use as routine screening while offering significant advantages in accuracy and capabilities compared to the stethoscope. More accurate and expensive medical diagnostics can be recommended based on the results of the 4D sound map, as needed, helping to avoid costs where more expensive medical diagnostics may not otherwise be necessary.

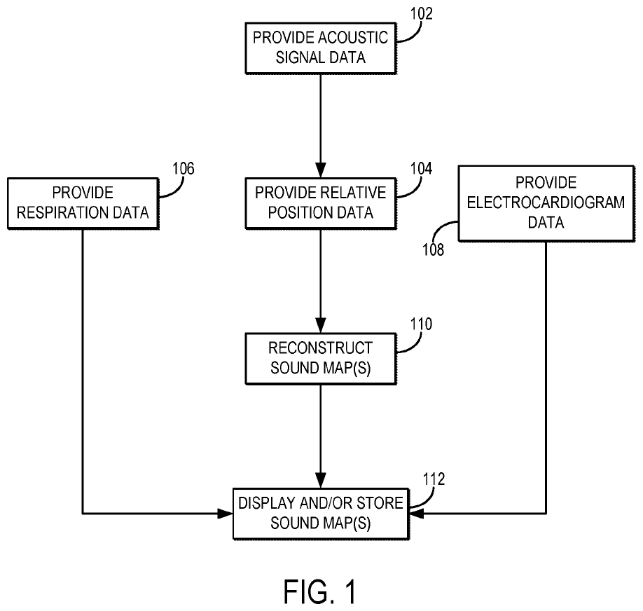

Referring now to FIG. 1, a flowchart is illustrated as setting forth the steps of an example method for generating a sound map from acoustic signals recorded using an array of microphones or other acoustic measurement devices or sensors. Acoustic signal data acquired from a subject are provided to a computer system, as indicated at step 102. Providing the acoustic signal data can include retrieving previously acquired data from a memory or other data storage device or medium. Additionally or alternatively, providing the acoustic signal data can include acquiring such data from a subject and providing the data to the computer system for processing. In either case, the acoustic signal data are acquired using an array or microphones or other acoustic measurement devices or sensors.

In general, the acoustic signal data include sound recordings measured at each of the microphones or other acoustic measurement devices or sensors in the array. The relative position of these microphones or other acoustic measurement devices or sensors can be used to compute a spatial distribution of acoustic sources within the subject. Thus, relative position data are provided to the computer system, as indicated at step 104. As noted, these relative position data generally indicate the relative positioning between the microphones or other acoustic measurement devices or sensors in the array. The relative position data may be provided by retrieving such data from a memory or other data storage device or medium, or by acquiring such data and providing it to the computer system.

As one example, the relative position data can include previously known spatial relationships between each microphone or other acoustic measurement device or sensor in the array. As another example, the relative position data can be acquired based on optical, radio frequency (“RF”), or other tracking of the microphones or other acoustic measurement devices or sensors in the array.

In some instances, the relative position data can be measured using a conductive elastic band that is coupled to the microphones or other acoustic measurement devices or sensors. As one example, the conductive elastic band can be a band composed of graphene elastic motion sensors, which by changing the resistance due to the amount of stretch in the band provides information about relative poisoning of the microphones or other acoustic measurements devices or sensors. Examples of such graphene elastic bands are described by C. Boland, et al., in “Sensitive, High-Strain, High-Rate Bodily Motion Sensors Based on Graphene-Rubber Composites,” ACS Nano, 2014; 8(9): 8819-8830.

Advantageously, an elastic band such as those described above can also act as respiration sensor strap. Thus, in some embodiments respiration data can be measured and provided to the computer system, as indicated at optional step 106.

The surface of the microphones or other acoustic measurement devices or sensors can in some instances be used as electrocardiogram sensors to provide more detailed information about heart rate and rhythm, as well as an indirect measurement of blood flow to the ventricles. In these instances, electrocardiogram data can also be measured and provided to the computer system, as indicated at option step 108.

Preferably, the array of microphones or other acoustic measurements devices or sensors can be overdetermined with redundant microphones such that distinct sound sources can be separated and such that the locations of the acoustic sources can be located using triangulation based on the relative times at which the various microphones detect the same sound signature. That is, if there is a sufficient number of microphones or other acoustic measurement devices or sensors with known relative positions, a unique sound distribution map can be generated based on the recordings from those microphones or other acoustic measurement devices or sensors over a small unit of time.

From the acoustic signal data and using the relative position data, a sound map depicting the spatiotemporal distribution of acoustic sources in the subject is reconstructed or otherwise generated, as indicated at step 110. The sound map can be generated using a suitable source localization algorithm. As one example, the source localization algorithm can include using beamforming. In one instance, the sound localization algorithm can include a beamforming-based acoustic imaging algorithm, such as the one described by H. Bing, et al., in “Three-Dimensional Localization of Point Acoustic Sources Using a Planar Microphone Array Combined with Beamforming,” R. Soc. Open Sci., 2018; 5:181407, which is herein incorporated by reference in its entirety.

The acoustic signal data may be transformed before or after reconstructing the sound map in order to extract frequency data, time data, both, or other data from the acoustic signal data. As an example, the acoustic signal data can be transformed using a wavelet transform, such as a continuous wavelet transform (“CWT”) to extract frequency and time information from the acoustic data. In some instances, the frequency information may be used to assist in the localization or characterization of acoustic sources in the subject. For example, the frequency information may indicate whether the acoustic source is associated with cardiac activity, respiration, or other physiological sources. For instance, by knowing the bandwidth of sound frequency that each organ can generate (and also knowing the sound frequency differences between healthy tissue and unhealthy tissue) a better estimation about the source of sounds can be achieved. Furthermore, using this information can help estimate the size and type of tissues that sit between the acoustic sensors and the acoustic source based on its attenuation coefficient and damping properties. In general using CWT combined with the original recorded sound can provide more accurate mapping about different physiological environments.

In some instances, the sound map generated or otherwise reconstructed at step 110 can be a 3D sound map depicting the spatial distribution of acoustic sources at a single time point. A plurality of such maps can be generated or reconstructed for different time points and combined to create a 4D sound map.

The sound map, or maps, can then be displayed or stored for later use, as indicated at step 112. For instance, a sound map can be displayed to a user, which may include displaying the sound map with a graphical user interface (“GUI”) that enables the user to interact with data in the sound map (e.g., retrieve or manipulate values in the sound map). In those instances where respiration data, electrocardiogram data, or both, were also provided to the computer system, these data can also be displayed to the user. For instance, these data can be overlaid with the sound maps, or displayed adjacent the sound maps in the same GUI. It will be appreciated that other forms of combining and displaying the sound maps with respiration data and electrocardiogram data are also possible.

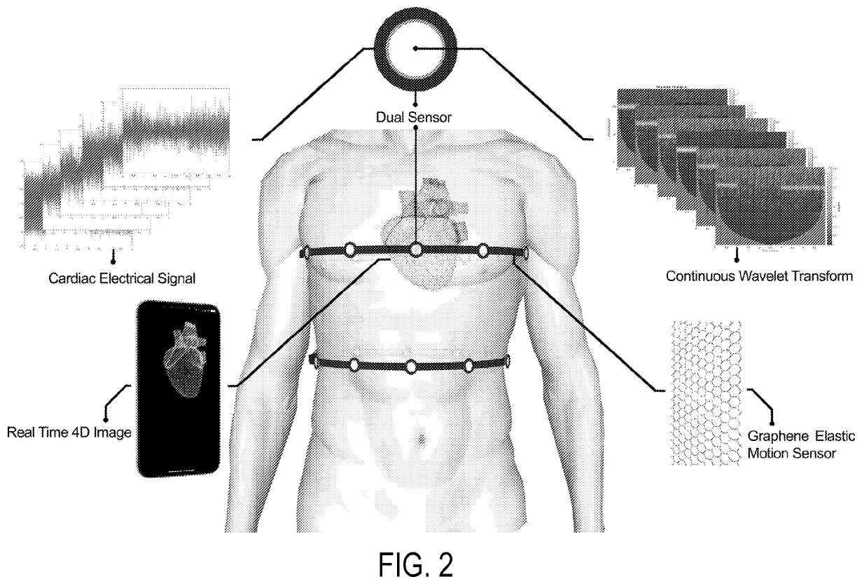

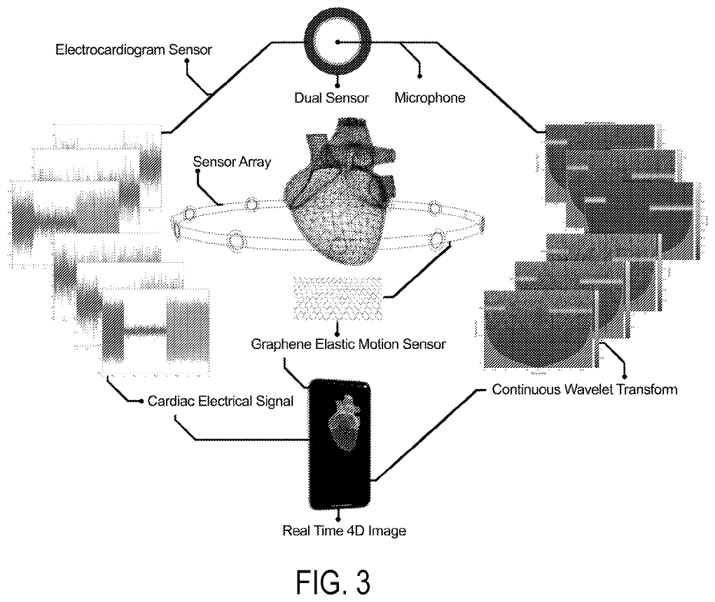

Referring now to FIGS. 2 and 3, an example of a system is shown for acquiring acoustic signal data from a subject and generating therefrom one or more sound maps, as described above. The system generally includes one or more arrays of acoustic sensors, which may include microphones or other acoustic measurements devices or sensors. These acoustic sensors can be coupled to one or more conductive bands, such as graphene elastic bands. The acoustic sensors can be dual sensors that also provide a measurement of cardiac electrical activity. The data collected from the sensors are provided to a computer system, which in some instances may include a smart phone or other portable computing device. Sound maps are reconstructed from the measured data and are displayed to a user.

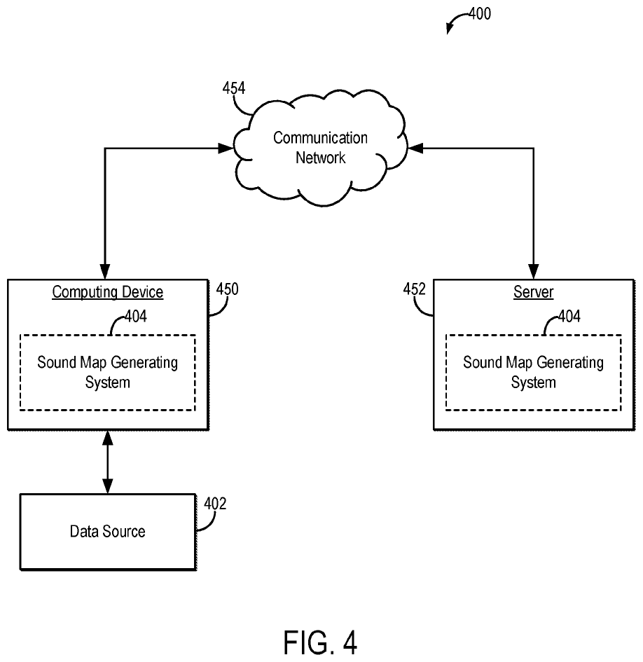

Referring now to FIG. 4, an example of a system 400 for generating sound maps, such as 4D sound maps, in accordance with some embodiments of the systems and methods described in the present disclosure is shown. As shown in FIG. 4, a computing device 450 can receive one or more types of data (e.g., acoustic signal data) from data source 402, which may be an acoustic signal data source. In some embodiments, computing device 450 can execute at least a portion of a sound map generating system 404 to generate sound maps (e.g., 4D sound maps) from data received from the data source 402.

Additionally or alternatively, in some embodiments, the computing device 450 can communicate information about data received from the data source 402 to a server 452 over a communication network 454, which can execute at least a portion of the sound map generating system 404 to generate sound maps (e.g., 4D sound maps) from data received from the data source 402. In such embodiments, the server 452 can return information to the computing device 450 (and/or any other suitable computing device) indicative of an output of the sound map generating system 404 to generate sound maps (e.g., 4D sound maps) from data received from the data source 402.

In some embodiments, computing device 450 and/or server 452 can be any suitable computing device or combination of devices, such as a desktop computer, a laptop computer, a smartphone, a tablet computer, a wearable computer, a server computer, a virtual machine being executed by a physical computing device, and so on. The computing device 450 and/or server 452 can also reconstruct images from the data.

In some embodiments, data source 402 can be any suitable source of acoustic signal data, such as an array of microphones or other acoustic signal measurement devices, another computing device (e.g., a server storing acoustic signal data), and so on. In some embodiments, data source 402 can be local to computing device 450. For example, data source 402 can be incorporated with computing device 450 (e.g., computing device 450 can be configured as part of a device for capturing, scanning, and/or storing acoustic signal data). As another example, data source 402 can be connected to computing device 450 by a cable, a direct wireless link, and so on. Additionally or alternatively, in some embodiments, data source 402 can be located locally and/or remotely from computing device 450, and can communicate data to computing device 450 (and/or server 452) via a communication network (e.g., communication network 454).

In some embodiments, communication network 454 can be any suitable communication network or combination of communication networks. For example, communication network 454 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links, Bluetooth links, cellular links, and so on.

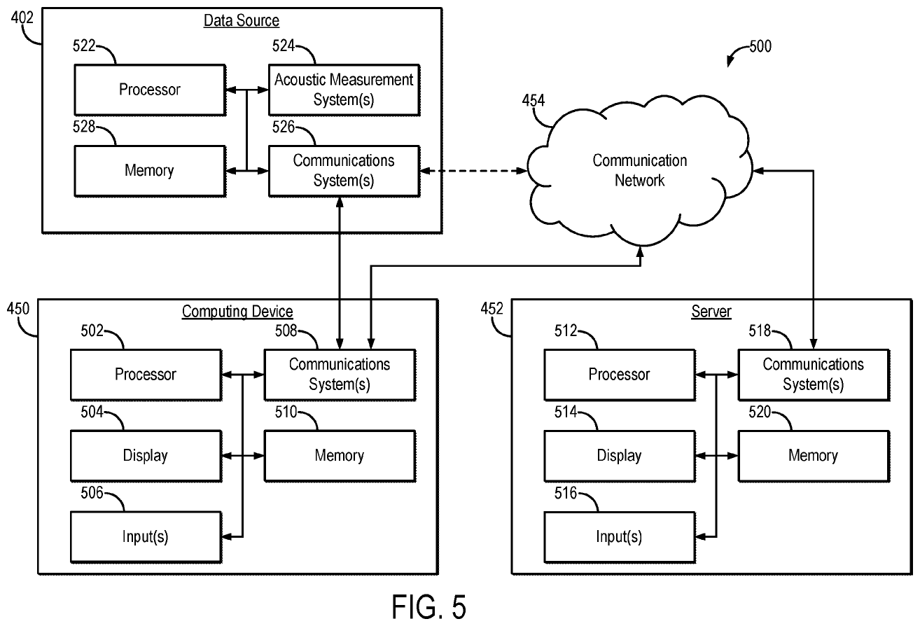

Referring now to FIG. 5, an example of hardware 500 that can be used to implement data source 402, computing device 450, and server 454 in accordance with some embodiments of the systems and methods described in the present disclosure is shown. As shown in FIG. 5, in some embodiments, computing device 450 can include a processor 502, a display 504, one or more inputs 506, one or more communication systems 508, and/or memory 510. In some embodiments, processor 502 can be any suitable hardware processor or combination of processors, such as a central processing unit (“CPU”), a graphics processing unit (“GPU”), and so on. In some embodiments, display 504 can include any suitable display devices, such as a computer monitor, a touchscreen, a television, and so on. In some embodiments, inputs 506 can include any suitable input devices and/or sensors that can be used to receive user input, such as a keyboard, a mouse, a touchscreen, a microphone, and so on.

In some embodiments, communications systems 508 can include any suitable hardware, firmware, and/or software for communicating information over communication network 454 and/or any other suitable communication networks. For example, communications systems 508 can include one or more transceivers, one or more communication chips and/or chip sets, and so on. In a more particular example, communications systems 508 can include hardware, firmware and/or software that can be used to establish a Wi-Fi connection, a Bluetooth connection, a cellular connection, an Ethernet connection, and so on.

In some embodiments, memory 510 can include any suitable storage device or devices that can be used to store instructions, values, data, or the like, that can be used, for example, by processor 502 to present content using display 504, to communicate with server 452 via communications system(s) 508, and so on. Memory 510 can include any suitable volatile memory, non-volatile memory, storage, or any suitable combination thereof. For example, memory 510 can include RAM, ROM, EEPROM, one or more flash drives, one or more hard disks, one or more solid state drives, one or more optical drives, and so on. In some embodiments, memory 510 can have encoded thereon, or otherwise stored therein, a computer program for controlling operation of computing device 450. In such embodiments, processor 502 can execute at least a portion of the computer program to present content (e.g., images, user interfaces, graphics, tables), receive content from server 452, transmit information to server 452, and so on.

In some embodiments, server 452 can include a processor 512, a display 514, one or more inputs 516, one or more communications systems 518, and/or memory 520. In some embodiments, processor 512 can be any suitable hardware processor or combination of processors, such as a CPU, a GPU, and so on. In some embodiments, display 514 can include any suitable display devices, such as a computer monitor, a touchscreen, a television, and so on. In some embodiments, inputs 516 can include any suitable input devices and/or sensors that can be used to receive user input, such as a keyboard, a mouse, a touchscreen, a microphone, and so on.

In some embodiments, communications systems 518 can include any suitable hardware, firmware, and/or software for communicating information over communication network 454 and/or any other suitable communication networks. For example, communications systems 518 can include one or more transceivers, one or more communication chips and/or chip sets, and so on. In a more particular example, communications systems 518 can include hardware, firmware and/or software that can be used to establish a Wi-Fi connection, a Bluetooth connection, a cellular connection, an Ethernet connection, and so on.

In some embodiments, memory 520 can include any suitable storage device or devices that can be used to store instructions, values, data, or the like, that can be used, for example, by processor 512 to present content using display 514, to communicate with one or more computing devices 450, and so on. Memory 520 can include any suitable volatile memory, non-volatile memory, storage, or any suitable combination thereof. For example, memory 520 can include RAM, ROM, EEPROM, one or more flash drives, one or more hard disks, one or more solid state drives, one or more optical drives, and so on. In some embodiments, memory 520 can have encoded thereon a server program for controlling operation of server 452. In such embodiments, processor 512 can execute at least a portion of the server program to transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone), and so on.

In some embodiments, data source 402 can include a processor 522, one or more acoustic measurement systems 524, one or more communications systems 526, and/or memory 528. In some embodiments, processor 522 can be any suitable hardware processor or combination of processors, such as a CPU, a GPU, and so on. In some embodiments, the one or more acoustic measurement systems 524 are generally configured to acquire acoustic signal data and can include an array of microphones or other suitable acoustic measurement devices. Additionally or alternatively, in some embodiments, one or more acoustic measurement systems 524 can include any suitable hardware, firmware, and/or software for coupling to and/or controlling operations of an array of microphones or other suitable acoustic measurement devices. In some embodiments, one or more portions of the one or more acoustic measurement systems 524 can be removable and/or replaceable.

Note that, although not shown, data source 402 can include any suitable inputs and/or outputs. For example, data source 402 can include input devices and/or sensors that can be used to receive user input, such as a keyboard, a mouse, a touchscreen, a microphone, a trackpad, a trackball, and so on. As another example, data source 402 can include any suitable display devices, such as a computer monitor, a touchscreen, a television, etc., one or more speakers, and so on.

In some embodiments, communications systems 526 can include any suitable hardware, firmware, and/or software for communicating information to computing device 450 (and, in some embodiments, over communication network 454 and/or any other suitable communication networks). For example, communications systems 526 can include one or more transceivers, one or more communication chips and/or chip sets, and so on. In a more particular example, communications systems 526 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 528 can include any suitable storage device or devices that can be used to store instructions, values, data, or the like, that can be used, for example, by processor 522 to control the one or more acoustic measurement systems 524, and/or receive data from the one or more acoustic measurement systems 524; to reconstruct images from acoustic signal data; present content (e.g., images, a user interface) using a display; communicate with one or more computing devices 450; and so on. Memory 528 can include any suitable volatile memory, non-volatile memory, storage, or any suitable combination thereof. For example, memory 528 can include RAM, ROM, EEPROM, one or more flash drives, one or more hard disks, one or more solid state drives, one or more optical drives, and so on. In some embodiments, memory 528 can have encoded thereon, or otherwise stored therein, a program for controlling operation of data source 402. In such embodiments, processor 522 can execute at least a portion of the program to generate images, transmit information and/or content (e.g., data, images) to one or more computing devices 450, receive information and/or content from one or more computing devices 450, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for generating a sound map that depicts a spatial distribution of acoustic sources within a subject, the steps of the method comprising:
(a) acquiring acoustic signal data from a subject using an array of acoustic sensors coupled to a surface of the subject and arranged around an anatomical region-of-interest;
(b) providing relative position data that indicate a relative position of acoustic sensors in the array of acoustic sensors; and
(c) reconstructing from the acoustic signal data and using the relative position data, a sound map that depicts a spatial distribution of acoustic sources in the subject.

2. The method of claim 1, wherein the sound map is reconstructed using a source localization algorithm implemented with a hardware processor and a memory.

3. The method of claim 2, wherein the source localization algorithm includes a beamforming algorithm.

4. The method of claim 1, wherein step (c) includes reconstructing a plurality of sound maps each corresponding to a different time point and combining the plurality of sound maps to generate a four-dimensional sound map that depicts a spatiotemporal distribution of the acoustic sources in the subject.

5. The method of claim 4, wherein the sound map depicts the spatiotemporal distribution of the acoustic sources as sound intensity in time and space being encoded by a spectrum of colors.

6. The method of claim 4, further comprising generating spectral data by applying a wavelet transform to the acoustic signal data and using the spectral data when reconstructing the sound map in order to guide determination of the acoustic sources.

7. The method of claim 6, wherein the spectral data is used to guide the determination of the acoustic sources by associating the spectral data with bandwidths of sound frequencies associated with different organs.

8. The method of claim 1, wherein the relative position data are provided by a conductive elastic band coupled to the array of acoustic sensors.

9. The method of claim 1, wherein the relative position data are provided by tracking positions of each acoustic sensor in the array of acoustic sensors.

10. The method of claim #, wherein tracking the positions of each acoustic sensor in the array of acoustic sensors comprises at least one of optical or radio frequency (RF) tracking.

11. A sound map generating system, comprising:
a sensor array configured to be worn around an anatomical region-of-interest, comprising:
a plurality of acoustic sensors;
an elastic motion sensor coupling each of the acoustic sensors to form the sensor array;
a computing device in communication with the sensor array and being configured to:
receive acoustic signal data from the plurality of acoustic sensors;
receive relative position data from the elastic motion sensor; and
reconstruct from the acoustic signal data using the relative position data, a sound map that depicts a spatial distribution of acoustic sources in a subject wearing the sensor array.

12. The sound map generating system of claim 11, wherein each of the plurality of acoustic sensors further comprise an electrocardiogram sensor and wherein the computing device is further configured to receive and store cardiac electrical signal data from each electrocardiogram sensor.

13. The sound map generating system of claim 12, wherein:
the computing device further comprises a display; and
the computing device generates a graphical user interface (GUI) on the display, the GUI comprising a visual depiction of the sound map and the cardiac electrical signal data.

14. The sound map generating system of claim 11, wherein the elastic motion sensor comprises a graphene elastic motion sensor to which each of the plurality of acoustic sensors is coupled.

15. The sound map generating system of claim 11, wherein the elastic motion sensor is sized to be worn around a chest of a subject and the computing device is further configured to process the relative position data to determine an expansion and contraction of the elastic motion sensor during respiration, thereby generating respiration data that are stored by the computing device.

16. The sound map generating system of claim 15, wherein:
the computing device further comprises a display; and
the computing device generates a graphical user interface (GUI) on the display, the GUI comprising a visual depiction of the sound map and the respiration data.

17. The sound map generating system of claim 11, wherein each of the plurality of acoustic sensors comprises a microphone.

18. The sound map generating system of claim 11, wherein:
the computing device further comprises a display; and
the computing device generates a graphical user interface (GUI) on the display, the GUI comprising a visual depiction of the sound map.

19. The sound map generating system of claim 11, wherein the computing device comprises a mobile device that is in communication with the sensor array via a wireless connection.

20. The sound map generating system of claim 11, further comprising a second sensor array configured to be worn around a second anatomical region-of-interest, comprising:
a second plurality of acoustic sensors; and
a second elastic motion sensor coupling each of the second plurality of acoustic sensors to form the second sensor array.
